(19) United States
(12) Patent Application Publication
(10) Pub. No.: US 2015/0299751 A1
(43) Pub. Date: Oct. 22, 2015

(54) METHOD OF ENZYMATIC TREATMENT OF A SOLID LIGNOCELLULOSIC MATERIAL

(71) Applicants: INSTITUT NATIONAL POLYTECHNIQUE DE TOULOUSE, Toulouse (FR); CENTRO DE INVESTIGACIONES ENERGETICAS, MEDIOAMBIENTALES Y TECNOLOGICAS, Madrid (ES); UNIVERSIDAD NACIONAL AUTONOMA DE MEXICO, Mexico Distrito Federal (MX)

(72) Inventors: Gerard VILAREM, Balma (FR); Luc RIGAL, Saint-Jean (FR); Virginie VANDENBOSSCHE, SAINTE FOY D'AIGREFEUILLE (FR); Julien BRAULT, L'ISLE ADAM (FR); Martin HERNANDEZ LUNA, Tlaplan, Mexico D.F. (MX); Oscar HERNANDEZ MELENDEZ, Delegacion Iztacalco, Mexico D.F. (MX); Eduardo VIVALDO LIMA, Colonia Vertiz Narvarte, Mexico D.F. (MX); Eduardo BARZANA GARCIA, Mexico D.F. (MX); Mercedes BALLESTEROS, MADRID (ES); Aleta DUQUE, MADRID (ES); Paloma MANZANARES, MADRID (ES); Matti Samuel SIIKA-AHO, HELSINKI (FI); Jaana Maria UUSITALO, Espoo (FI); Julio MATA-SEGREDA, CEDROS DE MONTES DE OCA (CR); Stephane GUILLOUET, VALLEGUE (FR); Eric LOMBARD, GRENADE (FR); Xavier CAMELEYRE, CASTANET TOLOSAN (FR)

(21) Appl. No.: 14/405,791

(22) PCT Filed: Jun. 7, 2013
(86) PCT No.: PCT/FR2013/051317

§ 371 Date: Dec. 5, 2014

(30) Foreign Application Priority Data
Jun. 8, 2012 (FR) 1255394

(51) Int. Cl. C12P 19/14; C12P 7/10; C12P 19/02

(52) U.S. Cl. CPC C12P 19/14; C12P 19/02; C12P 7/10; C12P 2201/00; C12P 2203/00

(57) ABSTRACT

A method of treating a solid lignocellulosic material (10) in which the solid lignocellulosic material (10) is subjected to a treatment, called a mechano-chemical treatment (1), of mixing and chemical degradation of the solid lignocellulosic material (10) so as to form an intermediate composition having a hydrated lignocellulosic material whose enzymatic digestibility is increased relative to the digestibility of the solid lignocellulosic starting material (10), then; the hydrated lignocellulosic material is subjected to a treatment, called a mechano-chemical treatment (2), in which a dispersion is formed, called an aqueous dispersion, of the hydrated lignocellulosic material (10) in an aqueous composition, the aqueous dispersion including at least one enzyme for degrading the hydrated lignocellulosic material (10).

The invention relates to a method of enzymatic treatment of a solid lignocellulosic material. The invention relates to such a method for the preparation of a solution—in particular an aqueous solution—of saccharides and/or phenolic compounds. The invention also relates to the use of such a method for the preparation of such an aqueous solution of saccharides for the purpose of their fermentation—in particular alcoholic—for the production of ethanol.

In particular, the object of the invention is a method for the preparation of a solution of saccharides comprising at least one monosaccharide and/or at least one oligosaccharide and/or at least one polysaccharide having a degree of polymerization and/or molecular weight suitable for the formation of a solution.

Such a method of enzymatic treatment of a solid lignocellulosic material is used in the general field of exploitation of vegetable resources, such as vegetable waste—in particular vegetable waste produced by agriculture or the food processing industry—or such as crops of herbaceous plants intended for the production of biomass—in particular for the production of energy. Such a method is also used in the conversion of vegetable waste into materials which are no longer waste and which can be converted, for example, into ethanol by alcoholic fermentation.

A method of treatment of biomass for the production of fermentable sugars is known (US 2007/0031918), in which said biomass is treated with an aqueous solution of ammonia and, where appropriate, of a base in a manner such as to form a concentrated dispersion of the biomass in the ammonia, the pH of the biomass dispersion is adjusted to a value of pH 5 and the sugars of the biomass are hydrolyzed enzymatically by means of a cellulase for a duration of the order of 96 hours.

Such a method requires a step in which the biomass is subjected to reduced pressure in order to eliminate the ammonia from the reaction medium before the addition of cellulases. Such a step is complex to implement on an industrial scale. Such a method no longer allows rapid treatment of a biomass composition, in particular in less than 96 hours.

Samaniuk et al. (2011), “Bioresource technology, 102, 4489-4494”, also discloses a method in which crushed maize stems are subjected to a treatment in an acid medium and then to an enzymatic degradation treatment of the solid material obtained by this treatment in an acid medium. Such a method requires a preceding step of crushing of maize stems, a step of neutralization of the acid medium, a step of filtration/washing in vacuo of the neutralized medium suitable for the preparation of a solid material prior to its enzymatic treatment. Such a method is complex and is not suitable for implementation on an industrial scale.

The object of the invention is to remedy the disadvantages described above by proposing a method of enzymatic treatment of a solid lignocellulosic material for the production of a solution—in particular an aqueous solution—of saccharides and/or phenolic compounds which is rapid to implement. In particular, such a method allows a solution of saccharides and/or phenolic compounds to be obtained starting from a solid lignocellulosic material in less than one hour—in particular in a duration of the order of 10 min.

The object of the invention is also to propose a method of enzymatic treatment of a solid lignocellulosic material suitable for allowing the exploitation of vegetable resources (biomass) which are no longer waste but which are capable of being exploited, in particular in the form of phenolic compounds, saccharides produced from the hemicelluloses and the cellulose of the solid lignocellulosic material, for example in the form of monosaccharide(s), such as glucose, or in the form of its fermentation products—for example in the form of ethanol.

The object of the invention is thus in particular, but not exclusively, a method of enzymatic treatment of a solid lignocellulosic material for the purpose of the production of ethanol and its use as a biofuel.

In particular, the object of the invention is to propose a method of enzymatic treatment of a solid lignocellulosic material which is available in a large quantity.

Another object of the invention is to propose such a method of enzymatic treatment of a solid lignocellulosic material which does not require refining of the cellulose produced from a vegetable resource before its conversion into solubilized saccharides and glucose for the purpose of its alcoholic fermentation.

The object of the invention is also such a method of enzymatic treatment of a solid lignocellulosic material in which no step of acid hydrolysis—in particular under the action of heat—capable of generating reaction by-products able to interfere with—or even inhibit—a subsequent step of alcoholic fermentation of glucose is carried out.

The object of the invention is also to achieve all these objects at a lower cost by proposing such a method of enzymatic treatment of a solid lignocellulosic material which is easy to implement and which requires no modification of pre-existing industrial installations.

The object of the invention is also such a method of enzymatic treatment of a solid lignocellulosic material suitable for allowing a rapid preparation of a solution of saccharides and/or phenolic compounds.

The object of the invention is also such a method of enzymatic treatment—in particular enzymatic degradation—of a solid lignocellulosic material of improved efficiency.

To this effect, the invention relates to a method of treatment of a solid lignocellulosic material in which said solid lignocellulosic material is subjected to a treatment, called mechano-chemical treatment, of kneading of said solid lignocellulosic material and chemical degradation of said solid lignocellulosic material, in which the solid lignocellulosic material is brought into contact with an alkaline solution in a manner such as to form an intermediate composition comprising a lignocellulosic material, called hydrated lignocellulosic material, having an enzymatic digestibility which is increased with respect to the digestibility of the starting solid lignocellulosic material; and then

the hydrated lignocellulosic material is subjected to a treatment, called mechano-enzymatic treatment, in which a dispersion, called aqueous dispersion, of the hydrated lignocellulosic material in an aqueous composition is formed, said aqueous dispersion comprising at least one enzyme for degradation of said hydrated lignocellulosic material; and
the mechano-enzymatic treatment is carried out by kneading said aqueous dispersion in a kneading reactor suitable for allowing a succession of mechanical phases of compression, expansion and shearing of the aqueous dispersion in a manner such as to form an aqueous solution, called hydrolysis solution, of hydrolysis products of said solid lignocellulosic material.

In all that follows, the expression “lignocellulosic material” means any natural material comprising cellulose, hemicellulose or lignins. In particular, such a solid lignocellulosic material according to the present invention comprises a proportion by weight of cellulose of between 20% and 100% of the dry matter. It can therefore be pure cellulose. The treatment according to the invention of such a pure cellulose allows the formation of a solution of saccharides and, where appropriate, glucose.

Such a method of mechano-enzymatic treatment of a solid lignocellulosic material is suitable for allowing the production of an aqueous solution of at least one product of enzymatic degradation of one of the constituents of the solid lignocellulosic material. In particular, such a method is suitable for allowing the production of an aqueous solution of saccharides and/or D-glucose starting from said solid lignocellulosic material.

In such a method of treatment of a solid lignocellulosic material according to the invention, the solid lignocellulosic material is first subjected to a treatment, called mechano-chemical treatment:
of kneading of said solid lignocellulosic material—in particular in a kneading reactor suitable for allowing a succession of mechanical phases of compression, expansion and shearing of the solid lignocellulosic material; and
of chemical degradation of said solid lignocellulosic material;
   in which the solid lignocellulosic material is brought into contact with an alkaline solution in a manner such as to form an intermediate composition comprising a lignocellulosic material, called hydrated lignocellulosic material, having an enzymatic digestibility—in particular of cellulose—which is increased with respect to the digestibility—in particular of cellulose—of the starting solid lignocellulosic material.

Advantageously, in a method according to the invention the solid lignocellulosic material is subjected to a first mechano-chemical treatment of kneading and chemical degradation of said solid lignocellulosic material, in which the solid lignocellulosic material is brought into contact with the alkaline solution in a manner such as to form, by impregnation and swelling of the solid lignocellulosic material, an intermediate composition comprising a solid lignocellulosic material, called hydrated lignocellulosic material, having a sensitivity to enzymatic hydrolysis which is increased with respect to the sensitivity to enzymatic hydrolysis of the starting solid lignocellulosic material. The hydrated lignocellulosic material is then subjected, where appropriate after neutralization of the intermediate composition, to mechano-enzymatic treatment.

The hydrated lignocellulosic material is distinguished by the fact that it comprises in particular a proportion of type II cellulose with respect to type I cellulose which is increased with respect to the proportion of type II cellulose with respect to type I cellulose of the starting solid lignocellulosic material. The type I and type II cellulose are quantified by techniques known per se to the person skilled in the art, in particular by x-ray diffraction of the dried starting solid lignocellulosic material and hydrated lignocellulosic material.

Advantageously, the alkaline solution is an aqueous solution of at least one alkali metal hydroxide —in particular selected from the group formed by sodium hydroxide and potassium hydroxide. Advantageously, the pH of the alkaline solution is greater than 7.5. In one method according to the invention the pH of the alkaline solution can have any value, for example of the order of 8 or greater than 13.

Advantageously and according to the invention, the mechano-chemical treatment is carried out at a temperature of between 50° C. and 150° C.

In such a method of treatment of a solid lignocellulosic material according to the invention, a concentrated aqueous dispersion of said hydrated lignocellulosic material is formed. Such an aqueous dispersion comprises at least one enzyme for degradation of said hydrated lignocellulosic material which is suitable for allowing a molecular cleavage of at least one of the constituents of the hydrated lignocellulosic material.

In fact, the inventors have found that, contrary to acknowledged prejudice in the state of the art, it is possible to carry out an enzymatic attack on a solid hydrated lignocellulosic material in the form of an aqueous dispersion directly in a kneading reactor. In particular, the inventors have observed that such an enzymatic attack on the solid hydrated lignocellulosic material remains very efficient in spite of the elevated concentration of said solid hydrated lignocellulosic material in the aqueous dispersion, in spite of the mechanical conditions of successive compression/expansion and of shearing and of the temperature to which the aqueous dispersion comprising the enzyme(s) for degradation of said solid hydrated lignocellulosic material is subjected in the kneading reactor.

Advantageously, such a method according to the invention allows an enzymatic degradation of a solid lignocellulosic material to be obtained in a kneading reactor for a short duration of treatment (in particular a duration of less than 1 hour), whereas enzymatic treatments of the state of the art require a duration of treatment of greater than one hour—in particular greater than several hours.

In fact, the invention is contrary to the prejudices of the state of the art, according to which in order to carry out an enzymatic digestion of a substrate:
it is appropriate to form a solution—and not a dispersion—comprising the enzyme and the substrate under concentration conditions of said substrate which are low and suitable for limiting the inhibition of the enzyme by excess substrate;
it is appropriate not to subject the medium comprising the substrate to temperature and pressure constraints which are unusual for the use of enzymes.

The inventors have thereby observed that, surprisingly, it is possible to carry out an enzymatic hydrolysis—in particular a partial hydrolysis or a total hydrolysis—of a solid lignocellulosic material—in particular of cellulose, lignins and hemicelluloses of said solid lignocellulosic material—in a concentrated medium and under kneading conditions—in particular of successive compression/expansion and of shearing—and temperature conditions which are not described in the state of the art.

Advantageously, in a method according to the invention, in addition to the hydrolysis solution a solid residue which is capable of being subjected to a fermentation treatment suitable for the production of a quantity of ethanol is formed, the fermentation treatment of the solid residue giving a yield, expressed by the weight of ethanol produced during the fermentation treatment with respect to the weight of the solid residue, which is greater than the yield of fermentation of the starting solid lignocellulosic material carried out under the same conditions.

Advantageously and according to the invention, the aqueous dispersion has a ratio between the weight of the aqueous composition of the aqueous dispersion and the weight of the dry matter of said solid lignocellulosic material of the aqueous dispersion of between 1 and 4.

The weight of dry matter of the solid lignocellulosic material is determined by a method known per se to the person skilled in the art and in which said lignocellulosic material, exposed beforehand to a drying treatment at a temperature of the order of 103° C. for a duration necessary to obtain a substantially constant weight of said lignocellulosic material, is weighed.

Advantageously and according to the invention, the aqueous dispersion is obtained by optional addition of degradation enzyme(s) to the solid lignocellulosic material. In one method according to the invention it is possible to prepare beforehand an aqueous solution of the degradation enzyme and then to add said aqueous solution of degradation enzyme to the solid lignocellulosic material. In a second variant of a method according to the invention the aqueous dispersion of the solid lignocellulosic material is first prepared and a quantity of degradation enzyme is then added to said aqueous dispersion.

Advantageously and according to the invention, the solid lignocellulosic material comprises:
a proportion by weight of cellulose, expressed in dry weight of cellulose and dry weight of said solid lignocellulosic material, of between 20% and 99%, in particular between 20% and 98%, preferably between 20% and 90%, in particular between 30% and 60%;
a proportion by weight of hemicelluloses, expressed in dry weight of hemicelluloses and dry weight of the solid lignocellulosic material, of between 15% and 50%;
a proportion by weight of lignins, expressed in dry weight of lignins and dry weight of the lignocellulosic material, of between 0.1% and 30%.

Advantageously and according to the invention, the aqueous dispersion comprises a proportion by weight of degradation enzyme(s) with respect to the dry matter of the solid lignocellulosic material of between 1% and 20%.

Advantageously and according to the invention, the mechano-enzymatic treatment is carried out at a temperature of between 20° C. and 80° C.—in particular between 30° C. and 70° C., preferably between 45° C. and 55° C.

Advantageously and according to the invention, the kneading reactor is a twin-screw extruder.

In all that follows, the expression “twin-screw extruder” means a kneading device for a material—in particular a solid lignocellulosic material—said kneading device comprises two co-penetrating screws of direct pitch or inverted pitch driven in rotation in synchronism inside a chamber—in particular a tubular chamber—and having a bilobate form in transverse cross-section. Advantageously, the chamber and the co-penetrating screws are made of nitrided steel or of industrial alloy suitable for the operating conditions in particular of abrasiveness and corrosion. Each of the co-penetrating screws is formed from screw sections extending axially and successively over a grooved axis and axially serving successive treatment zones of the solid lignocellulosic material between a charging zone of the twin-screw extruder and an exit zone.

According to a preferred embodiment of a method according to the invention a twin-screw extruder is used as the kneading reactor. One advantage of using a twin-screw extruder as the kneading reactor arises from the speed of the treatment. In fact, depending on the conditions of implementation of the invention, a few minutes to a few dozen minutes of mechano-enzymatic treatment of a lignocellulosic material in a twin-screw extruder are sufficient to allow an at least partial hydrolysis of the solid lignocellulosic material.

The screw profile—defined by the linking, the form and the pitch of the elements making up the twin screws of the twin-screw extruder—and the speed of rotation of the twin screws are selected in order to obtain an effect of kneading and/or crushing and/or shearing of the aqueous dispersion of the lignocellulosic material and in order to provide a dwell time of said aqueous dispersion in the twin-screw extruder which is suitable for allowing an at least partial hydrolysis of the solid lignocellulosic material.

In one method according to the invention the solid lignocellulosic material undergoes partial destructuring during the mechano-enzymatic treatment of kneading and/or crushing and/or shearing of said solid cellulosic material but also a simultaneous enzymatic attack on the solid cellulosic material at a temperature of between 20° C. and 80° C.

The effect of kneading and/or crushing and/or shearing is obtained here due to a displacement maintained on at least one rigid mechanical device within and in contact with said solid cellulosic material. This displacement is called maintained because it continues during said mechano-enzymatic treatment: it can be continuous or discontinuous, it can be repeated in a regular or irregular manner.

For implementation of a mechano-enzymatic treatment according to the invention, such a twin-screw extruder has the benefit of allowing automation of all the steps and the realization of this treatment in a single and continuous operation.

The use of a twin-screw extruder advantageously allows a precise control of a large number of functional parameters of a method according to the invention (treatment temperature, mode and power of the compression, expansion and shearing, treatment time etc.). In fact, by modifying certain structural characteristics and/or certain functional characteristics of the twin-screw extruder, the operator intervenes in the method parameters.

By way of example of the structural characteristics of a twin-screw extruder at the level of which an operator may intervene and which are generally determined and fixed before it is started (and which usually cannot be modified during its functioning) there are mentioned in particular the profile of the screws, which essentially depends on the form of the thread of the screws (which can be, for example, trapezoidal, conjugated, single or double etc.) and on the screw pitch. Each of these screws can also have different sections (or segments), which may possibly differ from one another in the form of the thread and/or in the screw pitch. Some of these sections making up these screws may also possibly correspond to monolobate or bilobate kneading elements.

Among these functional characteristics of a twin-screw extruder used in a method according to the invention at the level of which an operator may intervene at any time (both when this equipment is stopped and when it is working) there may be mentioned:
the speed of rotation of the screws;
the temperature of the chamber (or casing); particular temperatures may be fixed in different modules extending longitudinally in this chamber.

The wide choice of structural characteristics and functioning of such a twin-screw extruder allows a great freedom of adjustment of the conditions of implementation of a method according to the invention, and in particular of definition of the optimum conditions (for example the temperature, the shearing force, the duration of the treatment) specific to each of the starting solid lignocellulosic materials selected. Advantageously, the twin screws of a twin-screw extruder used in the context of the invention may comprise at least two sections which differ in their screw profile.

These structural characteristics of the screws of the twin-screw extruder are suitable for allowing not only an advance of the solid lignocellulosic material of the aqueous dispersion along the body of the twin-screw extruder, but also the formation, during this advance of the cellulosic material, of zones of compression and/or kneading and/or crushing and/or shearing and/or swelling by hydration of the solid lignocellulosic material of the aqueous dispersion and an enzymatic attack on the solid lignocellulosic material of the aqueous dispersion.

In fact, contrary to prejudices known in the state of the art, the inventors have observed that in spite of the kneading and crushing—in particular successive compression/expansion and shearing—conditions encountered in the twin-screw extruder and the elevated concentration of substrate (solid lignocellulosic material) in the twin-screw extruder, the enzymes used in a mechano-enzymatic treatment according to the invention have an enzymatic activity which is maintained and in all cases is sufficient for rapid formation of solubilized saccharides while the mechano-enzymatic treatment is carried out in the twin-screw extruder.

Advantageously and according to the invention, the enzyme(s) for degradation of the solid lignocellulosic material are selected from the group formed by cellulases—in particular endoglucanases, exoglucanases and β-glucosidases.

Advantageously, the cellulase is selected from the group formed by enzymes of class E.C.3.2.1. —in particular of class E.C.3.2.1.4., of class E.C.3.2.1.6., of class E.C.3.2.1.21., of class E.C.3.2.1.92., of class E.C.3.2.1.176.—of the classification of enzymes. Advantageously, the aqueous dispersion of the solid lignocellulosic material comprises at least one lytic enzyme suitable for allowing rupture of at least one β-1,4 covalent bond between two D-glucoside units of the cellulose of the solid lignocellulosic material and its conversion into water-soluble saccharides.

Advantageously and according to the invention, at least one enzyme for degradation of the solid lignocellulosic material is selected from the group formed by enzymes for degradation of lignins.

Advantageously, the enzyme for degradation of lignin is selected from the group formed by laccases—in particular of class E.C.1.10.3.2. of the classification of enzymes—and peroxidases—in particular of class E.C.1.11.1.7. and of class E.C.1.11.1.9. of the classification of enzymes.

Advantageously and according to the invention, at least one enzyme for degradation of the solid lignocellulosic material is selected from the group formed by enzymes for degradation of hemicelluloses.

Advantageously, the enzyme for degradation of hemicelluloses is selected from the group formed by enzymes of class E.C.3.2.1.—in particular of class E.C.3.2.1.8., of class E.C.3.2.1.37. and of class E.C.3.2.1.89.—of the classification of enzymes.

Advantageously and according to the invention, during the mechano-enzymatic treatment the pH of the aqueous dispersion is maintained at a value of between pH 4 and pH 7—in particular between pH 5.0 and pH 6.0.

Advantageously and according to the invention, the solid lignocellulosic material is selected from the group formed by all or part of a maize plant—in particular from the group formed by shoots, stalks and stems—, cereal straw—in particular of barley—, waste from the production of tequila—in particular from Agave tequilana —, agave bagasse for the production of inulin, sugar-cane bagasse, a residue from the production of palm oil and a cake of an oleaginous plant.

Advantageously and according to the invention, the hydrolysis solution comprising solubilized saccharides—in particular monosaccharides and oligosaccharides—is subjected to a step of enzymatic fermentation of solubilized saccharides. Advantageously, a step of alcoholic fermentation of saccharides and D-glucose of the hydrolysis solution and its conversion into ethanol is carried out.

Advantageously and according to the invention, prior to the mechano-enzymatic treatment a volume of an aqueous solution, called acid solution, of at least one mineral acid suitable for reducing the pH of the intermediate composition and for the formation of a neutralized intermediate composition suitable for being subjected to the mechano-enzymatic treatment is added to the intermediate composition.

Advantageously, the pH of the neutralized intermediate composition is between pH 6 and pH 7.

Advantageously and according to the invention, the mechano-chemical treatment and the mechano-enzymatic treatment are carried out successively in the same kneading reactor—in particular the same twin-screw extruder. For this purpose, the twin-screw extruder reactor comprises:
an upstream zone of mechano-chemical treatment of the solid lignocellulosic material; and
an intermediate zone of washing and liquid/solid separation of an aqueous solution rich in salts produced by the neutralization and in hemicelluloses; and
a downstream zone of the mechano-enzymatic treatment.

Advantageously, the mechano-chemical treatment and the mechano-enzymatic treatment are carried out successively in different kneading reactors. In a particular embodiment of the invention, it is possible to use one and the same extruder for carrying out the mechano-chemical treatment of a solid lignocellulosic material in a first pass and the mechano-enzymatic treatment of the solid lignocellulosic material in a second pass.

Advantageously and according to the invention, the neutralized intermediate composition comprises a proportion by weight of dry matter of between 10% and 40%.

Advantageously and according to the invention, the neutralized intermediate composition is subjected to the mechano-enzymatic treatment without carrying out at any preceding step of fractionation—in particular liquid/solid separation—of the hydrated lignocellulosic material and an aqueous phase.

Advantageously and according to the invention, a step of liquid/solid separation of a solid residue of the solid lignocellulosic material and of the hydrolysis solution containing water-soluble saccharides is carried out.

Advantageously and according to the invention, a step of liquid/solid separation of a solid residue of the solid lignocellulosic material and of the hydrolysis solution comprising phenolic compounds produced from the lignins of the solid lignocellulosic material is carried out.

In a particular embodiment of the invention, the hydrolysis solution comprising the water-soluble saccharides is subjected to a step of fermentation—in particular an alcoholic fermentation—of the water-soluble saccharides for the purpose of the formation of ethanol.

The invention moreover relates to an aqueous solution of water-soluble saccharides and enzyme(s) for degradation of the solid lignocellulosic material obtained by a method according to the invention and suitable for being recyclable in a method of subsequent mechano-enzymatic treatment of a solid lignocellulosic material.

The object of the invention is also an aqueous solution of saccharides capable of being obtained by a method according to the invention. The object of the invention is also an aqueous solution of water-solube saccharides obtained directly by a method according to the invention.

The invention also relates to a method of the production of a composition of water-soluble saccharides and an aqueous solution of saccharides, wherein all or some of the characteristic mentioned above or below are combined.

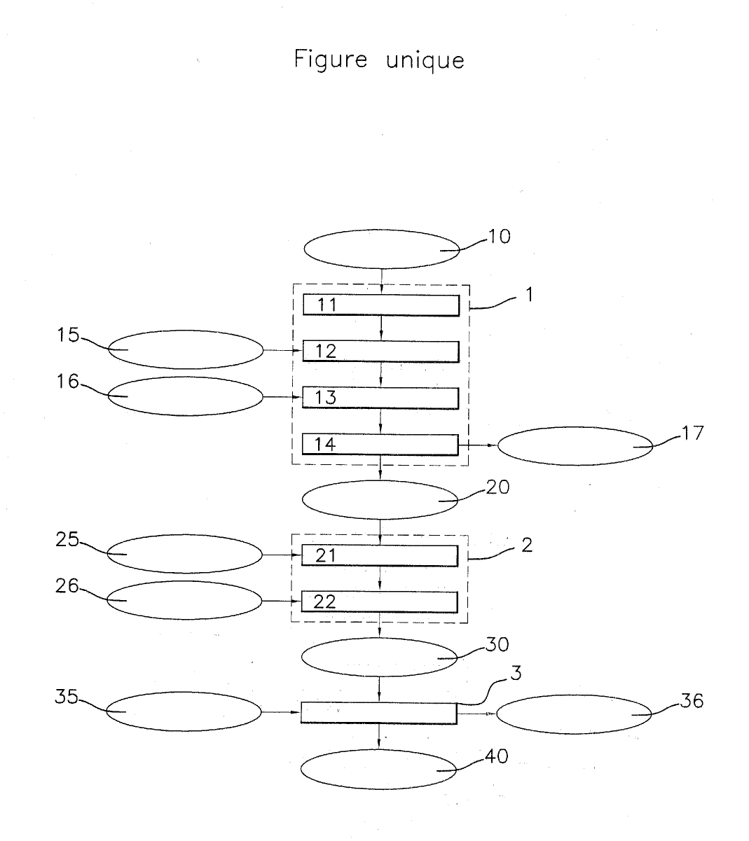

Other objects, characteristics and advantages of the invention will become apparent from reading the following description, which refers to the attached FIG. 1 illustrating an embodiment of a method according to the invention, and the examples given below solely by way of indication of an embodiment of a method according to the invention.

In a method of production of a composition of water-soluble saccharides according to the invention, shown in FIG. 1, a solid lignocellulosic material 10, in particular a solid lignocellulosic material 10 produced from biomass, is selected. Such a solid lignocellulosic material 10 advantageously has a dry matter content of between 15% and 99%.

Said solid lignocellulosic material 10 is introduced continuously into a first kneading device of a twin-screw extruder suitable for being able to subject the solid lignocellulosic material 10 successively to steps 11, 12, 13, 14 of a mechano-chemical treatment 1. The solid lignocellulosic material 10 is introduced into the first extruder with a flow rate suitable for the speed of rotation of the screws of the first twin-screw extruder and for the desired speed of advance of the solid lignocellulosic material 10 in the first twin-screw extruder.

The first twin-screw extruder is suitable for allowing an adjustment of the temperature of the solid lignocellulosic material 10 in each of steps 11, 12, 13, 14 of the mechano-chemical treatment 1. Step 11 of the mechano-chemical treatment 1 is a step of essentially mechanical treatment in which the solid lignocellulosic material 10 undergoes successive steps of compression, expansion and shearing in the twin-screw extruder. During this initial step 11 of the mechano-chemical treatment 1 the solid lignocellulosic material 10 is heated in a manner such as to reach a temperature of the order of 100° C. During step 12 of the mechano-chemical treatment 1 an alkaline solution 15—in particular of sodium hydroxide—is added to the solid lignocellulosic material 10 under mechanical treatment and at a temperature of the order of 100° C. Step 12 of addition of the alkaline solution 15 is suitable for allowing mixing of said alkaline solution 15 and the solid lignocellulosic material 10 and, where appropriate, a partial hydrolysis of the lignins of the solid lignocellulosic material 10.

At the end of this step 12 of alkaline hydrolysis an intermediate composition is formed comprising a solid lignocellulosic material, called hydrated lignocellulosic material, and having an enzymatic digestibility—in particular an enzymatic digestibility of the cellulose—which is increased with respect to the enzymatic digestibility—in particular with respect to the enzymatic digestibility of the cellulose—of the solid lignocellulosic material 10.

During the subsequent step 13 of the mechano-chemical treatment 1, a quantity of an acid solution 16 suitable for neutralizing the intermediate composition and for adjusting the pH of the intermediate composition to a value of the order of pH 6 to pH 7 is added continuously to said intermediate composition. Such a neutralization is carried out with an aqueous solution of a mineral acid, in particular with an aqueous solution of phosphoric acid in a manner such as to form a neutralized intermediate composition.

In the method according to the invention shown in FIG. 1, a step 14 of solid/liquid separation of a filtrate 17 rich in phenolic compounds and in hemicelluloses of the solid lignocellulosic material 10 and of a solid extrudate 20 enriched in hydrated cellulosic fibers having a crystalline type II cellulose content which is increased by 45% to 60% with respect to the starting solid lignocellulosic material 10 is then carried out. Such a step 14 of solid/liquid separation, however, is optional.

In a subsequent mechano-enzymatic treatment 2 of a solid extrudate 20 enriched in hydrates cellulosic fibers said solid extrudate 20 is introduced into a second twin-screw extruder or into an additional section of the same extruder. The second twin-screw extruder can be identical or similar to the first twin-screw extruder. At all events the second twin-screw extruder is suitable for allowing a mechanical treatment of the solid extrudate 20 by successive compression/expansion and by shearing. The solid extrudate 20 is introduced into the second twin-screw extruder with a flow rate suitable for the speed of rotation of the screws of the twin-screw extruder and for the desired speed of advance of the solid extrudate 20 in the second twin-screw extruder.

During the mechano-enzymatic treatment 2 a first step 21 of introduction of an aqueous solution 25 of at least one enzyme for hydrolysis of at least one constituent of the solid lignocellulosic material 10—in particular of cellulose—into the second twin-screw extruder is carried out. Such an introduction 21 of the enzymatic solution 25 into the second twin-screw extruder is carried out with a flow rate suitable for the speed of rotation of the screws of the second twin-screw extruder and for the desired speed of advance of the dispersion of the solid extrudate 20 in the enzymatic solution 25.

In addition, the temperature of the dispersion of the solid extrudate 20 in the enzymatic solution 25 is adjusted to a value suitable for allowing an optimum enzymatic activity. At all events, in a method according to the invention the enzymatic solution 25 is introduced into the second twin-screw extruder containing a concentrated solid extrudate 20. According to the method shown in FIG. 1 it is possible to proceed to an additional step 22 of introduction of an aqueous solution 26 of at least one enzyme for hydrolysis of cellulose. Such an introduction of the enzymatic solution 26 is carried out with a flow rate suitable for the speed of rotation of the screws of the second twin-screw extruder and for the desired speed of advance of the dispersion of the solid extrudate 20 in the enzymatic solution. It is possible for the enzymatic solutions 25, 26 to have the same enzymatic composition or different enzymatic compositions. It is possible to carry out the successive steps of hydrolysis at the same temperature or at different temperatures, each temperature being selected according to the optimum hydrolysis temperature of each enzymatic composition. At the end of this mechano-enzymatic treatment 2 a hydrolysate 30 comprising a solid fraction 40 comprising insoluble cellulose fibers, water-soluble cellulose fibers, saccharides, enzymes of the enzymatic solutions 25, 26 and, where appropriate, phenolic compounds of degradation of lignins and products of degradation of the hemicelluloses of the solid lignocellulosic material 10 is formed.

In the method according to the invention shown in FIG. 1, the hydrolysate 30 is subjected to a step 3 of liquid/solid separation and washing of said solid fraction 40 and an aqueous solution 35. In particular, this step 3 of liquid/solid separation and washing is carried out in a third twin-screw extruder or in an additional section of the same twin-screw extruder comprising a filtration module suitable for allowing a separation of the solid fraction 40 of the hydrolysate 30 and an aqueous liquid filtrate 36 comprising water-soluble cellulose fibers, saccharides, at least a part of the enzymes of the enzymatic solutions 25, 26 and, where appropriate, phenolic compounds produced by the degradation of the lignins and hemicelluloses and products of degradation of the hemicelluloses. Such a step of liquid/solid separation, however, is optional. In addition, advantageously, it is possible to use a single twin-screw extruder having a succession of sections, each section being suitable for allowing a continuous treatment of the solid lignocellulosic material.

In a first variant, not shown, of a method according to the invention a treatment of reduction of the grain size distribution of the solid lignocellulosic material is carried out prior to the mechano-chemical treatment. This step of reduction of the grain size distribution of the solid lignocellulosic material is carried out by any treatment known to the person skilled in the art, for example in a crusher, a chopping machine or any other device for reduction of the grain size distribution.

In a second variant, not shown, of a method according to the invention the aqueous liquid filtrate 36 obtained during the step of liquid/solid separation is collected, said aqueous liquid filtrate 36 comprising water-soluble cellulose fibers, saccharides, enzymes of the enzymatic solutions and, where appropriate, phenolic compounds produced from the degradation of the lignins and the products of degradation of the hemicelluloses, and this aqueous liquid filtrate comprising enzymes of the enzymatic solution(s) is recycled into the twin-screw extruder during a subsequent step of mechano-enzymatic treatment according to the invention. The fraction 40 can also be subjected to a second treatment cycle repeating all of the steps described above.

In a third variant, not shown, of a method according to the invention, a mechano-enzymatic treatment of a solid lignocellulosic material is carried out by introducing said lignocellulosic material directly into a twin-screw extruder. During the mechano-enzymatic treatment of said solid lignocellulosic material a first step of introduction of an aqueous solution of at least one enzyme for hydrolysis of at least one constituent of the solid lignocellulosic material—in particular of cellulose—is carried out. Such an introduction of the enzymatic solution into the twin-screw extruder is carried out with a flow rate suitable for the speed of rotation of the screws of the twin-screw extruder and for the desired speed of advance of the dispersion of the solid lignocellulosic material in the enzymatic solution.

### EXAMPLE 1

### Treatment of a Lignocellulosic Material Formed from Industrial Co-Products of Sweet Corn

A lignocellulosic material formed from non-exploited parts of sweet corn plants, in particular the stripped stalks and the shoots obtained after harvesting the maize cobs is collected. In this example the sweet corn co-product was subjected beforehand to a thermal dehydration treatment in a manner such as to obtain a dry matter content of 92% and allowing preservation thereof. A step of crushing of this sweet corn co-product is carried out in a VS 1 hammer mill (ELECTRA, Poudenas, France) equipped with a 2 mm selection grating. The characteristics of the dehydrated sweet corn co-product (cut shoots and cobs) are described below:

1 kg of sweet corn co-product (dry matter) comprises:
0.96 kg of organic matter;
0.39 kg of cellulose;
0.36 kg of hemicelluloses;
0.04 kg of lignins; and
0.06 kg of organic matter soluble in hot water.

The crushed sweet corn co-product is introduced continuously into a CLEXTRAL BC 45 twin-screw extruder with a flow rate of introduction of the dry matter of said crushed sweet corn co-product into the extruder of 11.4 kg/h.

### 1) Mechano-Chemical Treatment

A first mechano-chemical treatment of the crushed sweet corn co-product is carried out in a CLEXTRAL BC 45 twin-screw extruder (CLEXTRAL SA, Firminy, France) in which two parallel and identical screws of constant thread reach rotate at the same time and at the same speed in the bore of a fixed bilobate sheath. In such a twin-screw extruder the two parallel screws are of the “co-penetrating” type and are suitable for subjecting the crushed sweet corn co-product to mechanical work of shearing and mixing by means of successive sequences of compression, expansion and shearing.

The configuration of the CLEXTRAL BC 45 twin-screw extruder is described by way of non-limiting example in Table 1 below.

TABLE 1
Module

| | no. 1 | | no. 2 | |
|---|---|---|---|---|
| Heating | no | | no | |
| Temperature | ambient | | ambient | |
| Screw/disc | T2F | C2F | C2F | C2F |
| Length of section, cm | 10 | 10 | 10 | 10 |
| Screw pitch | 66 mm | 50 mm | 33 mm | 33 mm |
| (mm)/angle | | | | |

| | no. 3 | | no. 4 | | |
|---|---|---|---|---|---|
| Heating | yes | | yes | | |
| Temperature | 50° C. | | 102° C. | | |
| Length of section, cm | 10 | 5 | 10 | 10 | 5 |
| Screw/disc | BB | C2F | C2F | CF2C | C2F |
| Screw pitch | 90° | 33 mm | 25 mm | −25 mm | 33 mm |
| (mm)/angle | | | | | |

| | no. 5 | | | no. 6 | | no. 7 | | |
|---|---|---|---|---|---|---|---|---|
| Heating | yes | | | yes | | yes | | |
| Temperature | 102° C. | | | 53° C. | | 62° C. | | |
| Length of | 5 | 10 | 5 | 10 | 10 | 10 | 5 | 5 |
| section, cm | | | | | | | | |
| Screw/disc | C2F | BB | C2F | C2F | C2F | CF2C | C2F | C2F |
| Screw pitch | 33 | 90° | 33 | 33 mm | 25 mm | −25 mm | 33 mm | 25 mm |
| (mm)/angle | mm | | mm | | | | | |

In the configuration of a treatment device described in Table 1 and used in a method according to the invention, the CLEXTRAL BC 45 twin-screw extruder comprises seven modules linked linearly to one another in succession. It comprises:
a first module (module no. 1) extending from the charging zone and comprising:
   a section with a trapezoidal screw having a double thread of type T2F and having a screw pitch of 66 mm suitable for allowing introduction of the sweet corn co-product and conveying thereof; and
   a section with a conjugated screw having a double thread of type C2F and having a screw pitch of 50 mm suitable for allowing conveying of the sweet corn co-product;
a second module (module no. 2) extending downstream of module no. 1 and comprising:
   a section with a conjugated screw having a double thread of type C2F and having a screw pitch of 33 mm suitable for allowing conveying and compression of the sweet corn co-product;
   a second section with a conjugated screw having a double thread of type C2F and having a screw pitch of 33 mm suitable for allowing conveying and compression of the sweet corn co-product and a liquid composition intake;
a third module (module no. 3) thermoregulated at 50° C. and extending downstream of the second module and comprising:
   a series of 10 bilobate kneading discs of type BB (MAL 2) mounted perpendicularly to the grooved axis and displaced at an angle of 90° with respect to one another, suitable for allowing mixing of the sweet corn co-product and for increasing its dwell time in the extruder;
   a section with a conjugated screw having a double thread of type C2F and having a screw pitch of 33 mm suitable for allowing conveying of the sweet corn co-product;
a fourth module (module no. 4) thermoregulated at 102° C., extending downstream of the third module and comprising:
   a section with a conjugated screw having a double thread of type C2F and having a screw pitch of 25 mm suitable for allowing conveying of the sweet corn co-product;
   a section with an inverted screw of the type CF2C having a screw pitch of −25 mm;
   a section with a conjugated screw having a double thread of type C2F and having a screw pitch of 33 mm suitable for allowing conveying of the sweet corn co-product; and
a fifth module (module no. 5) thermoregulated at 102° C., extending downstream of the fourth module and comprising:
   a liquid composition intake; and
   a first section with a conjugated screw having a double thread of type C2F and having a screw pitch of 33 mm suitable for allowing conveying of the sweet corn co-product;
   a series of 5 bilobate kneading discs of the type BB (MAL 2) mounted perpendicularly to the grooved axis and displaced at an angle of 90° with respect to one another, suitable for allowing mixing of the sweet corn co-product and for increasing its dwell time in the extruder;
   a second section with a conjugated screw having a double thread of type C2F and having a screw pitch of 33 mm suitable for allowing conveying of the sweet corn co-product;
a sixth module (module no. 6) equipped with a filtration grating, extending downstream of the fifth module and comprising:
   a section with a conjugated screw having a double thread of type C2F and having a screw pitch of 33 mm;
   an exit for the liquid composition (filtrate) extending from this C2F section;
   a section with a conjugated screw having a double thread of type C2F and having a screw pitch of 25 mm;
a seventh module (module no. 7) of an exit thermoregulated at 62° C., extending downstream of the sixth module and comprising:
   a section with an inverted screw of the type CF2C having a screw pitch of −25 mm for exerting pressure on the material; and
   a section with a conjugated screw having a double thread of type C2F and having a screw pitch of 33 mm suitable for allowing re-uptake of the sweet corn co-product downstream of the counter-thread;
   a section with a conjugated screw having a double thread of type C2F and having a screw pitch of 25 mm allowing evacuation of the sweet corn co-product towards the outside of the sheath.

Such a screw profile is selected to allow successively introduction of the sweet corn co-product into the twin-screw extruder and then introduction of the sodium hydroxide solution, mixing thereof with the sweet corn co-product, for control of a contact time between the sweet corn co-product and the sodium hydroxide, and then neutralization of the medium by incorporation of acid, and, where appropriate, liquid/solid separation by filtration of a portion of the solubilized compounds.

In such a device the sweet corn co-product progresses axially by passing from one screw to another following an “open eight” path between the charging zone and the exit zone for the neutralized intermediate composition. The speed of rotation of the screws is suitable for avoiding blocking of the twin-screw extruder. In this example, the speed of rotation of the screws is 110 rpm.

An aqueous alkaline solution formed from 4 g of sodium hydroxide (NaOH) in 100 g of solution is introduced continuously at the level of the fourth module at a flow rate of said alkaline solution of 24 kg/h. Such an addition of the aqueous alkaline solution to the sweet corn co-product allows an addition of 90 g of sodium hydroxide per kilogram of dry matter of the sweet corn co-product.

The profile of the twin-screw extruder is suitable for allowing successively introduction of sweet corn co-product into the twin-screw extruder, mechanical kneading thereof, and then introduction of the alkaline solution, mixing of the alkaline solution and the sweet corn co-product under mechanical stress and subsequent introduction of the acid neutralization solution.

An aqueous acid solution formed from 2.4 g of phosphoric acid (H₃PO₄) in 100 g of solution is introduced continuously at the level of the fifth module with a flow rate of said acid solution of 38 kg/h. Such an addition of the aqueous acid solution into the twin-screw extruder allows an addition of 80 g of phosphoric acid per kilogram of dry matter of the sweet corn co-product.

A step of liquid/solid separation is carried out by filtration of the neutralized intermediate composition suitable for formation of an aqueous filtrate which flows out through the liquid composition (filtrate) exit of the sixth module.

At the exit of the seventh module of the CLEXTRAL BC 45 twin-screw extruder at the end of the mechano-chemical treatment, a neutralized intermediate composition at pH 6 is collected.

1 kg of dry matter of this neutralized intermediate composition comprises:
0.93 kg of organic matter;
0.45 kg of cellulose;
0.28 kg of hemicelluloses;
0.04 kg of lignins; and
0.08 kg of organic matter soluble in hot water.

### 2) Mechano-Enzymatic Treatment

This neutralized intermediate composition is recovered and is introduced into a CLEXTRAL BC 21 twin-screw extruder for a mechano-enzymatic treatment.

The configuration of the CLEXTRAL BC 21 twin-screw extruder is described by way of non-limiting example in Table 2 below.

TABLE 2
Module

| | no. 8 | | no. 9 | |
|---|---|---|---|---|
| Heating | no | | yes | |
| Temperature | ambient | | 40° C. | |
| Screw/disc | T2F | C2F | C2F | C2F |
| Length of section, cm | 5 | 5 | 5 | 5 |
| Screw pitch | 50 mm | 50 mm | 33 mm | 33 mm |
| (mm)/angle | | | | |
| | no. 10 | | no. 11 | |
| Heating | yes | | yes | |
| Temperature | 40° C. | | 45° C. | |
| Length of section, cm | 5 | 5 | 5 | 5 |
| Screw/disc | C2F | BB | C2F | C2F |
| Screw pitch | 33 mm | 90° | 25 mm | 16 mm |
| (mm)/angle | | | | |

| | no. 12 | no. 13 | no. 14 |
|---|---|---|---|
| Heating | yes | yes | yes |
| Temperature | 50° C. | 50° C. | 62° C. |

| | | | | | | | | | | | |
|---|---|---|---|---|---|---|---|---|---|---|---|
| Length of | 0.4 | 2.5 | 5 | 2.5 | 0.4 | 2.5 | 4.25 | 1.75 | 2.5 | 2.5 | 5 |
| section, cm | | | | | | | | | | | |
| Screw/disc | A | BB | C2F | C2F | A | BB | C2F | C2F | MALO | C2F | C2F |
| Screw pitch | — | −45° | 25 | 16 | — | −45° | 25 | 16 | −45° | 25 | 33 |
| (mm)/angle | | | mm | mm | | | mm | mm | | mm | mm |

In the configuration of a treatment device described in Table 2 and used in a method according to the invention, the CLEXTRAL BC 21 twin-screw extruder comprises seven modules linked linearly to one another in succession. It comprises:
an eighth module (module no. 8) extending from the charging zone and comprising two successive sections with a trapezoidal screw having a double thread of type T2F and having a screw pitch of 50 mm suitable for allowing introduction of the neutralized intermediate composition and conveying thereof; and
a ninth module (module no. 9) thermoregulated at 40° C. extending downstream of the eighth module and comprising two sections with conjugated screws having a double thread of type C2F and having a screw pitch of 33 mm suitable for allowing conveying and compression of the neutralized intermediate composition and a liquid enzymatic composition intake;
a tenth module (module no. 10) thermoregulated at 40° C. extending downstream of the ninth module and comprising:
   a section with a conjugated screw having a double thread of type C2F and having a screw pitch of 33 mm suitable for allowing conveying of the neutralized intermediate composition;
   a series of 10 bilobate kneading discs of type BB (MAL 2) mounted perpendicularly to the grooved axis and displaced at an angle of 90° with respect to one another, suitable for allowing mixing of the neutralized intermediate composition and for increasing its dwell time in the extruder;
an eleventh module (module no. 11) thermoregulated at 45° C., extending downstream of the tenth module and comprising:
   a section with a conjugated screw having a double thread of type C2F and having a screw pitch of 25 mm suitable for allowing conveying of the neutralized intermediate composition;
   a section with a conjugated screw having a double thread of type C2F and having a screw pitch of 16 mm suitable for allowing conveying of the neutralized intermediate composition; and
a twelfth module (module no. 12) thermoregulated at 50° C., extending downstream of the eleventh module and comprising:
   a neutral ring; and
   a series of 5 bilobate kneading discs of type BB (MAL 2) mounted perpendicularly to the grooved axis and displaced at an angle of −45° with respect to one another, suitable for allowing mixing of the neutralized intermediate composition and for increasing its dwell time in the extruder; and
   a section with a conjugated screw having a double thread of type C2F and having a screw pitch of 25 mm suitable for allowing conveying of the neutralized intermediate composition; and
a thirteenth module (module no. 13) thermoregulated at 50° C. and extending downstream of the twelfth module and comprising:
   a section with a conjugated screw having a double thread of type C2F and having a screw pitch of 16 mm;
   a neutral ring; and
   a series of 5 bilobate kneading discs of type BB (MAL 2) mounted perpendicularly to the grooved axis and displaced at an angle of −45° with respect to one another; and
   a section with a conjugated screw having a double thread of type C2F and having a screw pitch of 25 mm;
   a section with a conjugated screw having a double thread of type C2F and having a screw pitch of 16 mm;
a fourteenth module (module no. 14) with an exit thermoregulated at 50° C., extending downstream of the thirteenth module and comprising:
   a series of monolobate kneading discs of type MALO (MAL 2) mounted perpendicularly to the grooved axis and displaced at an angle of −45° with respect to one another; and
   a section with a conjugated screw having a double thread of type C2F and having a screw pitch of 25 mm; and
   a section with a conjugated screw having a double thread of type C2F and having a screw pitch of 33 mm.

The neutralized intermediate composition is introduced into the CLEXTRAL BC 21 twin-screw extruder at the level of the charging zone of module no. 8 with a flow rate of dry matter of 1.5 kg/h. The speed of rotation of the screws of the CLEXTRAL BC 21 twin-screw extruder is 250 rpm.

A solution of enzymes (Sacchariseb C6, Advanced Enzyme Technologies Ltd, California, USA) in an aqueous 300 mM citrate buffer having an enzyme concentration of 3% (v/v) is introduced into the CLEXTRAL BC 21 twin-screw extruder. The flow rate of introduction of the solution of enzymes into the CLEXTRAL BC 21 twin-screw extruder is 1.8 kg of solution of enzymes per hour. The configuration of the CLEXTRAL BC 21 twin-screw extruder is suitable for allowing introduction of the neutralized intermediate composition into the twin-screw extruder, intimate mixing of said neutralized intermediate composition and the enzymatic composition and maintaining of contact between the neutralized intermediate composition and the solution of enzymes in a manner such as to allow at least partial hydrolysis of the organic matter—in particular the cellulose—of the solid lignocellulosic material.

At the end of the mechano-enzymatic treatment, at the exit of the seventh module of the CLEXTRAL BC 21 twin-screw extruder, a bioextruded composition (BEC) is collected.

1 kg of dry matter of this bioextruded composition (BEC) comprises:
0.895 kg of organic matter, 22 g of which are enzyme;
0.38 kg of cellulose;
0.17 kg of hemicelluloses;
0.04 kg of lignins; and
0.15 kg of organic matter soluble in hot water.

Under the operating conditions of this example of treatment of sweet corn co-products, the mechano-enzymatic treatment allows the hydrolysis of 10% of the cellulose of the neutralized intermediate composition and 37% of the hemicelluloses of the neutralized intermediate composition. It also allows the quantity of organic matter soluble under the influence of heat to be doubled.

In addition, the fermentation of 1 kg of bioextruded composition (BEC) leads, without additional addition of enzyme, to the formation of 120 g of ethanol, corresponding to:
56% of the maximum fermentation potential of all the hexoses of the bioextruded composition (BEC); and to
49% of the maximum fermentation potential of the hexoses of the starting sweet corn co-product.

### 3) Second Cycle of Successive Mechano-Chemical and Mechano-Enzymatic Treatments

At the end of the step of mechano-enzymatic treatment, an additional treatment of twin-screw extrusion is carried out, during which the bioextruded composition (BEC) is introduced into a CLEXTRAL BC 21 twin-screw extruder substantially similar to the twin-screw extruder used for the mechano-enzymatic treatment above and in which:
a device for introduction of water has been installed in the module 17; and
a filtration zone has been added at the level of a module 21 modified with respect to module 14 used during the mechano-enzymatic treatment, the configuration of which is described in Table 3 below.

TABLE 3
Module

| | no. 15 | | no. 16 | |
|---|---|---|---|---|
| Heating | no | | yes | |
| Temperature | ambient | | 50° C. | |
| Screw/disc | T2F | C2F | C2F | C2F |
| Length of section, cm | 5 | 5 | 5 | 5 |
| Screw pitch | 50 mm | 50 mm | 33 mm | 33 mm |
| (mm)/angle | | | | |
| | no. 17 | | no. 18 | |
| Heating | yes | | yes | |
| Temperature | 50° C. | | 50° C. | |
| Length of section, cm | 5 | 5 | 5 | 5 |
| Screw/disc | C2F | BB | C2F | C2F |
| Screw pitch | 33 mm | 90° | 25 mm | 16 mm |
| (mm)/angle | | | | |

| | no. 19 | no. 20 | no. 21 |
|---|---|---|---|
| Heating | yes | yes | yes |
| Temperature | 50° C. | 50° C. | 50° C. |

| | | | | | | | | | | |
|---|---|---|---|---|---|---|---|---|---|---|
| Length of | 0.4 | 2.5 | 5 | 2.5 | 0.4 | 2.5 | 4.25 | 1.75 | 5 | 5 |
| section, cm | | | | | | | | | | |
| Screw/disc | A | BB | C2F | C2F | A | BB | C2F | C2F | CF2C | C2F |
| Screw pitch | — | −45° | 25 | 16 | — | −45° | 25 | 16 | −33 | 33 |
| (mm)/angle | | | mm | mm | | | mm | mm | mm | mm |

Such a twin-screw extruder dedicated to additional twin-screw extrusion treatment is a CLEXTRAL BC 21 twin-screw extruder comprising seven modules linked linearly to one another in succession. The CLEXTRAL BC 21 twin-screw extruder of the additional extrusion treatment comprises:
a module (module no. 15) equivalent to module no. 8; and
a module (module no. 16) equivalent to module no. 9 above, thermoregulated at 53° C. and extending downstream of module no. 15; and
a module (module no. 17) equivalent to module no. 10 above, thermoregulated at 55° C. and extending downstream of module no. 16;
a module (module no. 18) equivalent to module no. 11 above, thermoregulated at 50° C. and extending downstream of module no. 17;
a module (module no. 19) equivalent to module no. 12 above, thermoregulated at 53° C. and extending downstream of module no. 18;
a module (module no. 20) equivalent to module no. 13 above, extending downstream of module no. 19 and comprising a fluid exit equipped with a device for solid/liquid separation by filtration having a mesh diameter of 500 μm; and
a module (module no. 21) thermoregulated at 56° C. and extending downstream of module no. 20 and comprising:
   a section with a conjugated screw having a double inverted thread of type CF2C and having a screw pitch of −33 mm; and
   a section with a conjugated screw having a double thread of type C2F, and having a screw pitch of 33 mm.

The speed of rotation of the screws of the additional extrusion extruder is 250 rpm. The flow rate of introduction of the dry matter of the bioextruded composition (BEC) into the additional extruder is 2.7 kg/h. The flow rate of introduction of water at the level of the aqueous liquid composition intake (module no. 16) is 7 kg/h). The liquid/solid weight ratio of the bioextruded composition (BEC) in the additional extruder is 5.2.

At the exit of module no. 21 of the twin-screw extruder and at the end of the treatment by washing, a washed intermediate composition is collected.

1 kg of said washed intermediate material comprises:
0.95 kg of organic matter; of which
0.49 kg is cellulose;
0.28 kg is hemicelluloses;
0.05 kg is lignins; and
0.02 kg is organic matter soluble in hot water.

At the end of the above step of washing, a second mechano-chemical treatment of the washed intermediate composition is carried out. The treatment is substantially identical to the treatment described in 1) of Example 1 and suitable for the CLEXTRAL BC 21 twin-screw extruder, the configuration of which is described by way of non-limiting example in Table 4 below.

Modules 24 and 26 are equipped with a liquid introduction means intended respectively for introduction of the alkaline solution and of the acid solution. Module 20 is a filtration module.

TABLE 4
Module

| | no. 22 | | no. 23 | |
|---|---|---|---|---|
| Heating | no | | yes | |
| Temperature | — | | 50° C. | |
| Screw/disc | T2F | T2F | C2F | C2F |
| Length of section, cm | 5 | 5 | 5 | 5 |
| Screw pitch | 50 mm | 50 mm | 33 mm | 33 mm |
| (mm)/angle | | | | |

| | no. 24 | | no. 25 | | | |
|---|---|---|---|---|---|---|
| Heating | yes | | yes | | | |
| Temperature | 100° C. | | 100° C. | | | |
| Length of section, cm | 5 | 5 | 5 | 2.5 | 0.4 | 2.5 |
| Screw/disc | C2F | BB | C2F | C2F | A | BB |
| Screw pitch | 33 mm | 90° | 25 mm | 16 mm | — | −45° |
| (mm)/angle | | | | | | |

| | no. 26 | | no. 27 | | | | no. 28 | |
|---|---|---|---|---|---|---|---|---|
| Heating | yes | | yes | | | | yes | |
| Temperature | 50° C. | | 50° C. | | | | 50° C. | |
| Length of | 2.5 | 5 | 0.4 | 2.5 | 4.25 | 1.75 | 5 | 5 |
| section, cm | | | | | | | | |
| Screw/disc | C2F | C2F | A | BB | C2F | C2F | CF2C | C2F |
| Screw pitch | 25 mm | 16 mm | — | −45° | 25 mm | 16 mm | −33 mm | 33 mm |
| (mm)/angle | | | | | | | | |

As in the first treatment, such a screw profile is selected to allow successively introduction of the washed intermediate composition into the twin-screw extruder and then introduction of the sodium hydroxide solution, mixing thereof with the intermediate, a contact time between the intermediate composition and the sodium hydroxide, and then neutralization of the medium by incorporation of acid, and liquid/solid separation by filtration of a portion of the solubilized compounds.

The speed of rotation of the screws is fixed at 250 rpm.

The washed intermediate composition is introduced continuously at the level of the first module of the CLEXTRAL BC 21 twin-screw extruder with a flow rate of dry matter of 0.99 kg/h. An aqueous alkaline solution formed from 4 g of sodium hydroxide (NaOH) in 100 g of solution is introduced continuously at the level of the twenty-second module at a flow rate of 4 kg/h. Such an addition of the aqueous alkaline solution into the washed intermediate composition allows an addition of 160 g of sodium hydroxide per kilogram of dry matter of the washed intermediate composition.

An aqueous acid solution formed from phosphoric acid (H₃PO₄) in water at 1.8% is introduced continuously at the level of the twenty-sixth module with a flow rate of said acid solution of 10 kg/h. Such an addition of the aqueous acid solution into the twin-screw extruder allows an addition of 180 g of phosphoric acid per kilogram of dry matter of the washed intermediate composition.

A step of liquid/solid separation is carried out by filtration of the neutralized intermediate composition suitable for formation of an aqueous filtrate which flows out through the liquid composition (filtrate) exit of the sixth module.

At the exit of the twenty-eighth module of the CLEXTRAL BC 21 twin-screw extruder at the end of the mechano-chemical treatment, a second neutralized intermediate composition at pH 6 is collected. 1 kg of dry matter of this second neutralized intermediate composition comprises 0.94 kg of organic matter, of which 0.60 kg is cellulose, 0.25 kg is hemicelluloses, 0.04 kg is lignin and 0.07 kg is organic matter soluble in hot water.

This second neutralized intermediate composition is recovered and is introduced into a CLEXTRAL BC 21 twin-screw extruder for a second mechano-enzymatic treatment carried out under operating conditions close to the mechano-enzymatic treatment in the CLEXTRAL BC 21 twin-screw extruder.

The configuration of the CLEXTRAL BC 21 twin-screw extruder used for this step of the second mechano-enzymatic treatment is described by way of non-limiting example in Table 5 below.

TABLE 5
Module

| | no. 29 | | no. 30 | |
|---|---|---|---|---|
| Heating | no | | yes | |
| Temperature | — | | 50° C. | |
| Screw/disc | T2F | T2F | C2F | C2F |
| Length of section, cm | 5 | 5 | 5 | 5 |
| Screw pitch | 50 mm | 50 mm | 33 mm | 33 mm |
| (mm)/angle | | | | |

| | no. 31 | | no. 32 | | | |
|---|---|---|---|---|---|---|
| Heating | yes | | yes | | | |
| Temperature | 50° C. | | 50° C. | | | |
| Length of section, cm | 5 | 5 | 5 | 2.5 | 0.4 | 2.5 |
| Screw/disc | C2F | BB | C2F | C2F | A | BB |
| Screw pitch | 33 mm | 90° | 25 mm | 16 mm | — | −45° |
| (mm)/angle | | | | | | |

| | no. 33 | | no. 34 | | | | no. 35 | |
|---|---|---|---|---|---|---|---|---|
| Heating | yes | | yes | | | | yes | |
| Temperature | 50° C. | | 50° C. | | | | 50° C. | |
| Length of section, cm | 2.5 | 5 | 0.4 | 2.5 | 4.25 | 1.75 | 5 | 5 |
| Screw/disc | C2F | C2F | A | BB | C2F | C2F | C2F | C2F |
| Screw pitch | 25 mm | 16 mm | — | −45° | 25 mm | 16 mm | 33 mm | 33 mm |
| (mm)/angle | | | | | | | | |

The second neutralized intermediate composition is introduced into the CLEXTRAL BC 21 twin-screw extruder at the level of the charging zone of module no. 29 with a flow rate of dry matter of 0.87 kg/h. The speed of rotation of the screws of the CLEXTRAL BC 21 twin-screw extruder is 250 rpm.

A solution of enzymes, Sacchariseb C6, in an aqueous 300 mM citrate buffer having an enzyme concentration of 3% (w/w) is introduced into the CLEXTRAL BC 21 twin-screw extruder. The flow rate of introduction of the solution of enzymes into the CLEXTRAL BC 21 twin-screw extruder is 2.3 kg of solution of enzymes per hour. At the end of the mechano-enzymatic treatment, at the exit of the thirty-fifth module of the CLEXTRAL BC 21 twin-screw extruder, a second bioextruded composition is collected. 1 kg of dry matter of this second bioextruded composition comprises 0.91 kg of organic matter, of which 46 g is enzymes, 0.52 kg is cellulose, 0.01 kg is hemicelluloses, 0.03 kg is lignin and 0.25 kg is organic matter soluble in hot water.

Under the operating conditions of this example of treatment of a biomass composition of industrial co-products of sweet corn, the second mechano-enzymatic treatment allows the hydrolysis of 94% of the hemicelluloses of the second neutralized intermediate composition, accompanied by an increase by a factor of 10 in the water-soluble organic matter in the second bioextruded composition.

The fermentation of 1 kg of the second bioextruded composition (without addition of additional enzymes) leads to the formation of 185 g of ethanol, corresponding to 62% of the maximum fermentation potential of all the hexoses of the second bioextruded composition, that is to say 54.3% of the maximum fermentation potential of all the hexoses of the initial industrial co-product of sweet corn. It should be noted that the washing filtrate comprises 18.5% of the maximum fermentation potential of the initial industrial co-product of sweet corn.

### EXAMPLE 2

### Treatment of a Biomass Composition Formed from Residues of the Production of Palm Oil

A biomass composition formed from non-exploited parts of the extraction of palm oil is collected. A step of crushing of palm residues during the extraction of palm oil is carried out in a VS 1 hammer mill (ELECTRA, Poudenas, France) equipped with a selection grating of 2 mm. 1 kg of dry matter of these palm residues comprises 0.97 kg of organic matter, of which 0.44 kg is cellulose, 0.24 kg is hemicelluloses, 0.19 kg is lignin and 0.04 kg is organic matter soluble in hot water.

These palm residues are subjected to the same succession of treatment as that applied to the industrial co-products of sweet corn mentioned in Example 1.

1) Mechano-Chemical Treatment

The first mechano-chemical treatment of the crushed palm residues is carried out in a CLEXTRAL BC 45 twin-screw extruder (CLEXTRAL SA, Firminy, France). The CLEXTRAL BC 45 twin-screw extruder comprises an opening for introduction of the palm residues at the level of module no. 1, a liquid alkaline composition intake at the level of module no. 2, a liquid acid composition intake at the level of module no. 6 and a filtration device at the level of module no. 6.

The holding temperature of the modules is given in Table 6 below.

TABLE 6

| | Module | | | | | | |
|---|---|---|---|---|---|---|---|
| | no. 1 | no. 2 | no. 3 | no. 4 | no. 5 | no. 6 | no. 7 |
| T ° C. | ambient | ambient | 40 | 100 | 50 | 50 | 70 |

The treatment conditions are described in Table 7 below.

TABLE 7

| | Flow | | | | | | |
|---|---|---|---|---|---|---|---|
| | rate | | NaOH | | | | |
| | of dry | | intake | | H₃PO₄ intake | | Enzymatic |
| | matter | Screw | Flow | Concen- | Flow | Concen- | solution |
| | intake | speed | rate | tration | rate | tration | Flow rate |
| Treatment | (kg/h) | (rpm) | (kg/h) | (%) | (kg/h) | (%) | (kg/h) |
| Mechano- | 8.5 | 200 | 27 | 4 | 63 | 1.8 | |
| chemical | | | | | | | |
| Mechano- | 1.5 | 200 | | | | | 5.6 |
| enzymatic | | | | | | | |

2) 1 kg of dry matter of this neutralized intermediate composition comprises 0.97 kg of organic matter, of which 0.53 kg is cellulose, 0.23 kg is hemicelluloses, 0.21 kg is lignins and 0.01 kg is organic matter soluble in hot water.

### 3) Mechano-Enzymatic Treatment

The neutralized intermediate composition is recovered and is introduced into a CLEXTRAL BC 21 twin-screw extruder for a mechano-enzymatic treatment.

The configuration of the CLEXTRAL BC 21 twin-screw extruder is described by way of non-limiting example in Table 8 below.

TABLE 8
Module

| | no. 8 | | no. 9 | |
|---|---|---|---|---|
| Peripheral | solid intake: material | | liquid intake: enzyme | |
| Heating | no | | yes | |
| Temperature | — | | 40° C. | |
| Screw/disc | T2F | T2F | C2F | C2F |
| Length of section, cm | 5 | 5 | 5 | 5 |
| Screw pitch | 50 mm | 50 mm | 33 mm | 33 mm |
| (mm)/angle | | | | |
| | no. 10 | | no. 11 | |
| Heating | yes | | yes | |
| Temperature | 40° C. | | 45° C. | |
| Length of section, cm | 5 | 5 | 5 | 5 |
| Screw/disc | C2F | BB | C2F | C2F |
| Screw pitch | 33 mm | 90° | 25 mm | 16 mm |
| (mm)/angle | | | | |

| | no. 12 | no. 13 | no. 14 |
|---|---|---|---|
| Heating | yes | yes | yes |
| Temperature | 50° C. | 50° C. | 62° C. |

| | | | | | | | | | | |
|---|---|---|---|---|---|---|---|---|---|---|
| Length of | 0.4 | 2.5 | 5 | 2.5 | 0.4 | 2.5 | 4.25 | 1.75 | 5 | 5 |
| section, cm | | | | | | | | | | |
| Screw/disc | A | BB | C2F | C2F | A | BB | C2F | C2F | C2F | C2F |
| Screw pitch | — | 90° | 25 | 16 | — | 90° | 25 | 16 | 33 | 16 |
| (mm)/angle | | | mm | mm | | | mm | mm | mm | mm |

The treatment conditions are described in Table 3 above. The enzymatic solution used is a solution of enzymes, Sacchariseb C6 (Advanced Enzyme Technologies Ltd), in an aqueous 300 mM citrate buffer having an enzyme concentration of 3% (w/w).

1 kg of dry matter of this bioextruded composition comprises 0.92 kg of organic matter, of which 83 g are enzyme, 0.38 kg is cellulose, 0.14 kg is hemicelluloses, 0.16 kg is lignins and 0.04 kg is organic matter soluble in hot water.

The fermentation of 1 kg of the bioextruded composition (without addition of additional enzymes) leads to the formation of 136 g of ethanol, corresponding to 51.5% of the maximum fermentation potential of all the hexoses of the bioextruded composition.

### EXAMPLE 3

### Treatment of a Biomass Composition of Barley Straw

A biomass composition formed from barley straw is collected. A step of crushing of the barley straw is carried out in a hammer mill equipped with a selection grating of 5 mm. 1 kg of dry matter of this barley straw comprises 0.93 kg of organic matter, of which 0.39 kg is cellulose, 0.26 kg is hemicelluloses, 0.16 kg is lignins and 0.1 kg is organic matter soluble in hot water.

### 1) Mechano-Chemical Treatment

A first mechano-chemical treatment of the crushed barley straw is carried out in a CLEXTRAL EV 25 twin-screw extruder (CLEXTRAL SA, Firminy, France).

The configuration of the extruder is described by way of non-limiting example in Table 9 below. The operating conditions used are described in Table 10.

TABLE 9
Module

| | no. 1 | | | | no. 2 | | | |
|---|---|---|---|---|---|---|---|---|
| Peripheral | solid intake: material | | | | | | | |
| | liquid intake: NaOH | | | | | | | |
| Heating | no | | | | yes | | | |
| Temperature | 35° C. | | | | 68° C. | | | |
| Screw/disc | C2F | C2F | C2F | BL22 | C2F | C2F | C2F | C2F |
| Length of | 31 | 31 | 18 | 12 | 31 | 31 | 25 | 18 |
| section, mm | | | | | | | | |
| Screw pitch | 31 mm | 31 mm | 18 mm | 45° | 31 mm | 31 mm | 25 mm | 18 mm |
| (mm)/angle | | | | | | | | |

| | no. 3 | | | | | no. 4 | | | |
|---|---|---|---|---|---|---|---|---|---|
| Heating | yes | | | | | yes | | | |
| Peripheral | | | | | | liquid intake: H₃PO₄ | | | |
| Temperature | 68° C. | | | | | 68° C. | | | |
| Screw/disc | BL22 | BL22 | C2F | C2F | C2F | CF1C | C2F | C2F | C2F |
| Length of | 25 | 25 | 31 | 12 | 12 | 12 | 25 | 25 | 25 |
| section, mm | | | | | | | | | |
| Screw pitch | 90 | 90 | 31 | 12 | 12 | −12 | 25 | 25 | 25 |
| (mm)/angle | | | mm | mm | mm | mm | mm | mm | mm |

| | no. 5 | | | | | | no. 6 | | | | |
|---|---|---|---|---|---|---|---|---|---|---|---|
| Peripheral | filter | | | | | | | | | | |
| Heating | no | | | | | | yes | | | | |
| Temperature | 60° C. | | | | | | 68° C. | | | | |
| Screw/disc | C2F | C2F | C2F | C2F | C2F | C2F | CF1C | C2F | C2F | C2F | C2F |
| Length of | 25 | 25 | 18 | 18 | 12 | 12 | 12 | 31 | 18 | 18 | 18 |
| section, mm | | | | | | | | | | | |
| Screw pitch | 25 | 25 | 18 | 18 | 12 | 12 | −12 | 31 | 18 | 18 | 18 |
| (mm)/angle | mm | mm | mm | mm | mm | mm | mm | mm | mm | mm | mm |

1 kg of dry matter of the neutralized intermediate composition comprises 0.95 kg of organic matter, of which 0.07 kg is organic matter soluble in hot water.

### 2) Mechano-Enzymatic Treatment

This neutralized intermediate composition is recovered and is introduced into a CLEXTRAL EV 25 twin-screw extruder for a mechano-enzymatic treatment.

The configuration of the extruder is described in Table 11 below. The operating conditions used are described in Table 10.

TABLE 11
Module

| | no. 7 | | | | no. 8 | | | |
|---|---|---|---|---|---|---|---|---|
| Peripheral | solid intake: material | | | | liquid intake: enzymatic solution | | | |
| Heating | no | | | | yes | | | |
| Temperature | — | | | | 50° C. | | | |
| Screw/disc | C2F | C2F | C2F | C2F | C2F | C2F | BL22 | BL22 |
| Length of | 31 | 31 | 25 | 25 | 18 | 12 | 25 | 25 |
| section, mm | | | | | | | | |
| Screw pitch | 31 mm | 31 mm | 25 mm | 25 mm | 18 mm | 12 mm | 90° | 90° |
| (mm)/angle | | | | | | | | |

| | no. 9 | | | | | no. 10 | | | | |
|---|---|---|---|---|---|---|---|---|---|---|
| Heating | yes | | | | | yes | | | | |
| Temperature | 50° C. | | | | | 50° C. | | | | |
| Screw/disc | C2F | C2F | C2F | C2F | C2F | BL22 | BL22 | C2F | C2F | C2F |
| Length of | 31 | 25 | 18 | 12 | 12 | 12 | 12 | 31 | 25 | 25 |
| section, mm | | | | | | | | | | |
| Screw pitch | 31 | 25 | 18 | 12 | 12 | −45° | −45° | 31 | 25 | 25 |
| (mm)/angle | mm | mm | mm | mm | mm | | | mm | mm | mm |

| | no. 11 | | | | | no. 12 | | | |
|---|---|---|---|---|---|---|---|---|---|
| Heating | no | | | | | yes | | | |
| Temperature | 50° C. | | | | | 50° C. | | | |
| Screw/disc | C2F | C2F | C2F | BL22 | BL22 | C2F | C2F | C2F | C2F |
| Length of | 18 | 18 | 12 | 25 | 25 | 31 | 25 | 25 | 18 |
| section, mm | | | | | | | | | |
| Screw pitch | 18 | 18 | 12 | −45° | −45° | 31 | 25 | 25 | 18 |
| (mm)/angle | mm | mm | mm | | | mm | mm | mm | mm |

The enzymatic solution used is a solution of enzymes (cellulase Cellic CTec2 and xylanase Cellic CTec2 in a proportion of 9:1 with respect to the protein content) in an aqueous 200 mM citrate buffer having an enzyme concentration of 2.5% (weight/weight). 1 kg of dry matter of this bioextruded composition comprises 0.95 kg of organic matter, of which 26 g are enzymes and 0.29 kg is organic matter soluble in hot water.

The fermentation of 1 kg of the bioextruded composition (without addition of additional enzymes) leads to the formation of 144 g of ethanol, corresponding to 60% of the maximum fermentation potential of all the hexoses of the bioextruded composition.

TABLE 10

| | Flow | | | | | | |
|---|---|---|---|---|---|---|---|
| | rate | | NaOH intake | | H₃PO₄ intake | | |
| | of dry | | | Concen- | | Concen- | Enzymatic |
| | matter | Screw | Flow | tration | Flow | tration | solution |
| | intake | speed | rate | (% | rate | (% | Flow rate |
| Treatment | (kg/h) | (rpm) | (kg/h) | (w/v)) | (kg/h) | (w/v)) | (kg/h) |
| Mechano- | 0.54 | 150 | 0.34 | 10 | 7.38 | 0.98 | |
| chemical | | | | | | | |
| Mechano- | 0.2 | 150 | | | | | 0.28 |
| enzymatic | | | | | | | |

### EXAMPLE 4

### Treatment of a Biomass Composition Formed from Industrial Co-Products of Agave Tequilana

A biomass composition formed from Agave tequilana Weber var. azul bagasse is collected. A step of crushing of this bagasse is carried out in a hammer mill equipped with a selection grating of 2 mm. 1 kg of dry matter of these residues of Agave plants comprises 0.96 kg of organic matter, of which 0.39 kg is cellulose, 0.17 kg is hemicelluloses, 0.19 kg is lignins and 0.22 kg is organic matter soluble in hot water.

Successive mechano-chemical treatment and mechano-enzymatic treatment are carried out continuously and successively on the crushed bagasse in a CLEXTRAL Evolum 25 twin-screw extruder (CLEXTRAL SA, Firminy, France).

The configuration of the CLEXTRAL Evolum 25 twin-screw extruder is described by way of non-limiting example in Table 12 below, and the operating conditions used are described in Table 13.

TABLE12
Module

| | no 1 | | | | | no. 2 | | | |
|---|---|---|---|---|---|---|---|---|---|
| Peripheral | solid intake: material | | | | | liquid intake: NaOH | | | |
| Heating | no | | | | | yes | | | |
| Temperature | 50° C. | | | | | 75° C. | | | |
| Length of section, | 12.5 | 31.25 | 25 | 18.75 | 12.5 | 25 | 25 | 31.25 | 25 |
| mm | | | | | | | | | |
| Screw/disc | C2F | C2F | C2F | C2F | C2F | BB | BB | C2F | C2F |
| Screw pitch | 12.5 | 31.25 | 25 | 18.75 | 12.5 | 45° | 90° | 31.25 | 25 |
| (mm)/angle | | | | | | | | | |

| | no. 3 | | | | no. 4 | | | | |
|---|---|---|---|---|---|---|---|---|---|
| Peripheral | | | | | liquid intake: H₃PO₄ | | | | |
| Heating | yes | | | | yes | | | | |
| Temperature | 75° C. | | | | 30° C. | | | | |
| Length of section, | 18.5 | 12 | 25 | 25 | 31.25 | 25 | 18.75 | 12.5 | 12.5 |
| mm | | | | | | | | | |
| Screw/disc | C2F | C2F | BB | BB | C2F | C2F | C2F | C2F | BB |
| Screw pitch | 18.5 | 12 | 45° | 90° | 31.25 | 25 | 18.75 | 12.5 | 90 |
| (mm)/angle | | | | | | | | | |

| | no. 5 | | | | no. 6 | | | | | | no. 7 | | | | | |
|---|---|---|---|---|---|---|---|---|---|---|---|---|---|---|---|---|
| Peripheral | filter | | | | liquid intake: enzyme | | | | | | | | | | | |
| Heating | no | | | | yes | | | | | | yes | | | | | |
| Temperature | 50° C. | | | | 40° C. | | | | | | 40° C. | | | | | |
| Length of | 31.25 | 25 | 18.75 | 18.75 | 12.5 | 25 | 12.5 | 12.5 | 31.25 | 25 | 25 | 18.75 | 18.75 | 12.5 | 12.5 | 12.5 |
| section, mm | | | | | | | | | | | | | | | | |
| Screw/disc | C2F | C2F | C2F | C2F | CF2C | CF2C | C2F | C2F | C2F | C2F | C2F | C2F | C2F | C2F | C2F | C2F |
| Screw pitch | 31.25 | 25 | 18.75 | 18.75 | −12.5 | −25 | 12.5 | 12.5 | 31.25 | 25 | 25 | 18.75 | 18.75 | 12.5 | 12.5 | 12.5 |
| (mm)/angle | | | | | | | | | | | | | | | | |
| | no. 8 | | | | | no. 9 | | | | no. 10 | | | | | | |
| Heating | yes | | | | | yes | | | | yes | | | | | | |
| Temperature | 40° C. | | | | | 40° C. | | | | 40° C. | | | | | | |
| Length of | 25 | 25 | 18.75 | 18.75 | 12.5 | 12.5 | 18.75 | 25 | 31.25 | 25 | 18.75 | 12.5 | 12.5 | 12.5 | 18.75 | 25 |
| section, mm | | | | | | | | | | | | | | | | |
| Screw/disc | C2F | C2F | C2F | C2F | C2F | C2F | C2F | C2F | C2F | C2F | C2F | C2F | C2F | C2F | C2F | C2F |
| Screw pitch | 25 | 25 | 18.75 | 18.75 | 12.5 | 12.5 | 18.75 | 25 | 31.25 | 25 | 18.75 | 12.5 | 12.5 | 12.5 | 18.75 | 25 |
| (mm)/angle | | | | | | | | | | | | | | | | |

The enzymatic solution used is made up of a mixture of Cellic CTec2 (Novozymes) and Viscozyme (Novozymes) in an aqueous 50 mM citrate buffer having an enzyme concentration of 0.2% (w/w).

1 kg of dry matter of this bioextruded composition comprises 0.93 kg of organic matter, of which 9 g are enzyme, 0.42 kg is cellulose, 0.10 kg is hemicelluloses, 0.16 kg is lignins and 0.25 kg is organic matter soluble in hot water. Under the operating conditions of this example, the treatment of the bagasse allows the hydrolysis of 22% of the cellulose and 58% of the hemicelluloses.

The fermentation of 1 kg of the bioextruded composition (without addition of additional enzymes) leads to the formation of 22.5 g of ethanol, corresponding to 9% of the maximum fermentation potential of all the hexoses of the bioextruded composition.

TABLE 13

| | Flow | | | | | | |
|---|---|---|---|---|---|---|---|
| | rate | | NaOH | | | | |
| | of dry | | intake | | H₃PO₄ intake | | Enzymatic |
| | matter | Screw | Flow | Concen- | Flow | Concen- | solution |
| | intake | speed | rate | tration | rate | tration | Flow rate |
| Treatment | (kg/h) | (rpm) | (kg/h) | (%) | (kg/h) | (%) | (kg/h) |
| Mechano- | 0.22 | 85 | 0.46 | 5 | 0.395 | 4.78 | 0.46 |
| chemical | | | | | | | |
| and | | | | | | | |
| mechano- | | | | | | | |
| enzymatic | | | | | | | |

1. A method of treatment of a solid lignocellulosic material (10) in which said solid lignocellulosic material (10) is subjected to a treatment, called mechano-chemical treatment (1), of kneading of said solid lignocellulosic material (10) and chemical degradation of said solid lignocellulosic material (10), in which the solid lignocellulosic material (10) is brought into contact with an alkaline solution in a manner such as to form an intermediate composition comprising a solid lignocellulosic material, called hydrated lignocellulosic material, having an enzymatic digestibility which is increased with respect to the digestibility of the starting solid lignocellulosic material (10); and then
the hydrated lignocellulosic material is subjected to a treatment, called mechano-enzymatic treatment (2), in which a dispersion, called aqueous dispersion, of the hydrated lignocellulosic material (10) in an aqueous composition is formed, said aqueous dispersion comprising at least one enzyme for degradation of said hydrated lignocellulosic material; and the mechano-enzymatic treatment (2) is carried out by kneading said aqueous dispersion in a kneading reactor suitable for allowing a succession of mechanical phases of compression, expansion and shearing of the aqueous dispersion in a manner such as to form an aqueous solution, called hydrolysis solution, of hydrolysis products of said solid lignocellulosic material (10).

2. A method as claimed in claim 1, wherein the aqueous dispersion has a ratio between the weight of the aqueous composition of the aqueous dispersion and the weight of the dry matter of said solid lignocellulosic material (10) of the aqueous dispersion of between 1 and 4.

3. A method as claimed in claim 1, wherein the aqueous dispersion is obtained by optional addition of degradation enzyme(s) to the solid lignocellulosic material (10).

4. A method as claimed in claim 1, wherein the solid lignocellulosic material (10) comprises:
a proportion by weight of cellulose, expressed in dry weight of cellulose and in dry weight of the solid lignocellulosic material (10), of between 20% and 99%;
a proportion by weight of hemicelluloses, expressed in dry weight of hemicelluloses and dry weight of the solid lignocellulosic material (10), of between 15% and 50%;
a proportion by weight of lignins, expressed in dry weight of lignins and dry weight of the solid lignocellulosic material (10), of between 0.1% and 30%.

5. A method as claimed in claim 1, wherein the aqueous dispersion comprises a proportion by weight of degradation enzyme(s) with respect to the dry matter of the solid lignocellulosic material (10) of between 1% and 20%.

6. A method as claimed in claim 1, wherein the mechano-enzymatic treatment (2) is carried out at a temperature of between 20° C. and 80° C.

7. A method as claimed in claim 1, wherein the kneading reactor is a twin-screw extruder.

8. A method as claimed in claim 1, wherein the enzyme(s) for degradation of the solid lignocellulosic material (10) is/are selected from the group formed by cellulases.

9. A method as claimed in claim 1, wherein at least one enzyme for degradation of the solid lignocellulosic material (10) is selected from the group formed by enzymes for degradation of lignins.

10. A method as claimed in claim 1, wherein at least one enzyme for degradation of the solid lignocellulosic material (10) is selected from the group formed by enzymes for degradation of hemicelluloses.

11. A method as claimed in claim 1, wherein the solid lignocellulosic material (10) is selected from the group formed by all or part of a maize plant, cereal straw, waste from the production of tequila, agave bagasse for the production of inulin, sugar-cane bagasse, a residue from the production of palm oil and a cake of an oleaginous plant.

12. A method as claimed in claim 1, wherein the hydrolysis solution comprising solubilized saccharides is subjected to a step of enzymatic fermentation of the solubilized saccharides.

13. A method as claimed in claim 1, wherein the mechano-chemical treatment (1) is carried out at a temperature of between 50° C. and 150° C.

14. A method as claimed in claim 1, wherein a volume of an aqueous solution, called acid solution, of at least one mineral acid suitable for reducing the pH of the intermediate composition and for the formation of a neutralized intermediate composition suitable for being subjected to the mechano-enzymatic treatment (2) is added to the intermediate composition.

15. A method as claimed in claim 2, wherein the aqueous dispersion is obtained by optional addition of degradation enzyme(s) to the solid lignocellulosic material (10).

16. A method as claimed in claim 2, wherein the solid lignocellulosic material (10) comprises:
a proportion by weight of cellulose, expressed in dry weight of cellulose and in dry weight of the solid lignocellulosic material (10), of between 20% and 99%;
a proportion by weight of hemicelluloses, expressed in dry weight of hemicelluloses and dry weight of the solid lignocellulosic material (10), of between 15% and 50%;
a proportion by weight of lignins, expressed in dry weight of lignins and dry weight of the solid lignocellulosic material (10), of between 0.1% and 30%.

17. A method as claimed in claim 2, wherein the aqueous dispersion comprises a proportion by weight of degradation enzyme(s) with respect to the dry matter of the solid lignocellulosic material (10) of between 1% and 20%.

18. A method as claimed in claim 2, wherein the mechano-enzymatic treatment (2) is carried out at a temperature of between 20° C. and 80° C.
